(19) United States
(12) Patent Application Publication
(10) Pub. No.: US 2008/0300622 A1
(43) Pub. Date: Dec. 4, 2008

(54) Atraumatic Hemostatic Clamp

(76) Inventor: LIN XU, Nanjing (CN)

Correspondence Address: GLOBAL IP SERVICES, 7285 W. Eagle Court, Winton, CA, 95388, US

(21) Appl. No.: 12/098,657

(22) Filed: Apr. 7, 2008

(30) Foreign Application Priority Data
May 29, 2007 (CN) 200720037895.1

(51) Int. Cl. A61B 17/28

(52) U.S. Cl. 606/207

(57) ABSTRACT

An atraumatic hemostatic clamp, being an artery/vein clamp in the field of medical instruments, mainly includes a left clamp body (1) and a right clamp body (2) which are joined by a hinge and may be divided into jaws (3) and handles (4) by the hinge. The jaws (3) have a curved configuration with ends extending upwardly. Serrated portions (8) with serrations projecting from engaging surfaces are formed on both ends of each of the jaws (3) of the left and right clamp bodies (1), (2). A groove (9) is formed between the serrated portions (8) on both ends. Therefore, this atraumatic hemostatic clamp has a simple structure and can provide a reliable grasp without causing trauma to bodily vessels, thus solving the problem of the conventional hemostatic clamp that vessel trauma may be easily caused.

### CROSS REFERENCE TO RELATED PATENT APPLICATION

This application claims the priority of the Chinese patent application No. 200720037895.1 filed on May 29, 2007, which is incorporated herein by reference.

### BACKGROUND OF THE INVENTION

1. Field of the Invention

The present invention relates to a medical instrument, especially a supplementary medical instrument for various surgical operations. In particular, the present invention relates to an atraumatic hemostatic clamp for occluding vein or artery to stop bleeding.

2. Related Art

Currently, in various surgical operations, e.g., pulmonary surgery, the pulmonary vessels must be occluded to prevent the flow of the blood for the convenience of operation. Thus, the pulmonary vascular clamps must be used. However, in order to provide a sufficient clamping force, usually, a tooth configuration is integrally formed on the surfaces of two jaws in contact with the vessels of the conventional pulmonary vascular clamp, so as to firmly grip the vessels. If the clamping force is too large, the tooth configuration on the jaws might cause perforation to the vessel walls, which leads to vessel trauma. Therefore, a higher requirement for the control of the clamping force during the operation is raised, which may increase the mental burden of doctors.

In addition, the width of the jaws of the conventional hemostatic clamp is generally smaller than 0.4 cm. The holding feeling and the strength of the conventional hemostatic clamp cannot meet actual requirements and thus need improvements.

### SUMMARY OF THE INVENTION

Accordingly, in order to optimize the unsatisfactory structure design and solve the problem of the conventional hemostatic clamp that vessel trauma or perforation of vein walls may be easily caused, the present invention is directed to an atraumatic hemostatic clamp capable of providing a reliable grasp without causing trauma to bodily vessels.

The technical scheme of the present invention is described as follow.

An atraumatic hemostatic clamp mainly includes a left clamp body 1 and a right clamp body 2 which are joined by a hinge and may be divided into jaws 3 and handles 4 by the hinge. The jaws 3 of the left and right clamp bodies 1, 2 have a curved configuration with ends extending upwardly. Serrated portions 8 with serrations projecting from the engaging surface are respectively formed on both ends of the jaw 3 of the left clamp body 1 and the jaw 3 of the right clamp body 2. A groove 9 is formed between the serrated portions 8 on both ends.

The serrated portions 8 on one jaw 3 have a single row configuration, and the serrated portions 8 on the other jaw 3 have a double row configuration. A slot 10 is formed between the double rows for insertion of the serrated portion 8 having the single row configuration.

One jaw 3 has a single groove 9 and the other jaw 3 has double grooves 9. A groove wall 11 formed between the double grooves 9 is opposite to the single groove 9, and can be inserted in the single groove 9 when the jaws 3 are closed.

A width L of the jaw 3 is in a range of 0.3 cm to 0.7 cm, and preferably, 0.3 cm to 0.5 cm.

The handles 4 of the left and right clamp bodies 1, 2 respectively have a clamping force adjustment mechanism acting in cooperation with each other. The adjustment mechanism is comprised of a ratchet means 6 formed on one of the left clamp body 1 and the right clamp body 2 and a pawl 7 formed on the other of the left clamp body 1 and the right clamp body 2. The ratchet means 6 has at least five ratchet teeth, and preferably, seven or more.

The present invention has the following advantages.

Instead of using the tooth configuration which is proved to have unsatisfactory vessel-occluding effect and even induce adverse influences based on plenty of clinical practices, the present invention adopts a structure with a smooth section having no teeth for gripping the vessel walls. Meanwhile, a groove structure lower than the clamping surface is formed on the clamping surface. Thus, a groove wall formed between the adjacent grooves on one clamping surface can be inserted in the groove on the other clamping surface, so as to ensure the clamping force. In addition, a contact deformation of the vessels gripped by the jaws is generated, and thus the vessels can be firmly gripped by the clamp. Further, the serrated configuration on two ends helps to prevent the slippage of the vessels. Also, the jaws are made thicker, such that this hemostatic clamp is safer and more convenient in use. In addition, the number of the adjustment teeth is increased to achieve a more precise adjustment of the clamping force and a more flexible manipulation.

### BRIEF DESCRIPTION OF THE DRAWINGS

The present invention will become more fully understood from the detailed description given herein below for illustration only, and thus are not limitative of the present invention, and wherein:

FIG. 1 is a schematic top view of a structure of a hemostatic clamp according to the present invention;

FIG. 2 is a schematic front view of the structure of the hemostatic clamp according to the present invention;

FIG. 3 is a schematic cross-sectional structural view taken along line A-A in FIG. 1; and

FIG. 4 is a schematic cross-sectional structural view taken along line B-B in FIG. 1.

### DETAILED DESCRIPTION OF THE INVENTION

Hereinafter, the present invention will be illustrated in the following embodiments with the accompanying drawings.

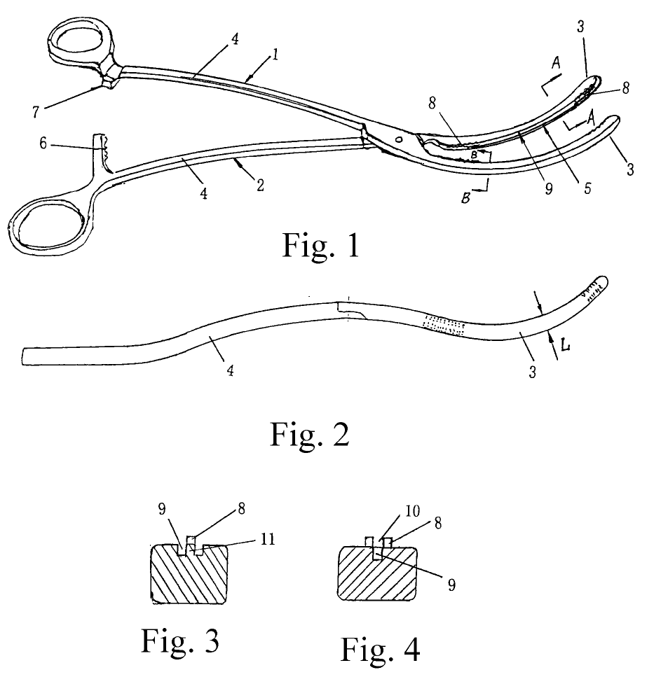

Refer to FIGS. 1 and 2.

As shown in FIG. 1, an atraumatic hemostatic clamp made of titanium alloy mainly includes a left clamp body 1 and a right clamp body 2 which are joined by a hinge and may be divided into jaws 3 and handles 4 by the hinge. The jaws 3 of the left and right clamp bodies 1, 2 have a curved configuration with ends extending upwardly. Serrated portion 8 with serrations projecting from the engaging surface are respectively formed on both ends of the jaw 3 of the left clamp body 1 and the jaw 3 of the right clamp body 2. A groove 9 is formed on a non-serrated section 5 between the serrated portions 8 on the two ends. The serrated portions 8 on one jaw 3 have a single row configuration (in FIG. 3). As shown in FIG. 4, the serrated portions 8 on the other jaw 3 have a double row configuration, and a slot 10 is formed between the double rows for insertion of the serrated portions 8 having the single row configuration. Further, one jaw 3 has a single groove 9 (in FIG. 4) and the other jaw 3 has double grooves 9 (in FIG. 3). A groove wall 11 formed between the double grooves 9 is opposite to the single groove 9, and the top of the groove wall 11 is in a same level with the top of the single groove 9 when the jaws 3 are closed. A width L of the jaw 3 is in a range of 0.4 cm to 0.7 cm, and preferably, 0.5 cm or above, as shown in FIG. 2. The handles 4 of the left and right clamp bodies 1, 2 respectively have a clamping force adjustment mechanism acting in cooperation with each other. The adjustment mechanism includes a ratchet means 6 formed on one of the left clamp body 1 and the right clamp body 2 and a pawl 7 formed on the other of the left clamp body 1 and the right clamp body 2. The ratchet means 6 has at least five ratchet teeth, and preferably, seven or more, as shown in FIG. 1.

What is claimed is:

1. An atraumatic hemostatic clamp comprising: a left clamp body (1) and a right clamp body (2) joined and divided into jaws (3) and handles (4) by a hinge; wherein the jaws (3) having a curved configuration with ends extending upwardly; serrated portions (8) with serrations projecting from engaging surfaces being formed on two ends of the jaw (3) of the left clamp body (1) and two ends of the jaw (3) of the right clamp body (2); and a groove (9) being formed between the serrated portions (8).

2. The atraumatic hemostatic clamp according to claim 1, wherein the serrated portions (8) on one jaw (3) have a single row configuration, the serrated portions (8) on the other jaw (3) have a double rows configuration, and a slot (10) is formed between the double rows for insertion of the serrated portions (8) having the single row configuration.

3. The atraumatic hemostatic clamp according to claim 1, wherein one jaw (3) has a single groove (9), the other jaw (3) has double grooves (9), and a groove wall (11) formed between the double grooves (9) is opposite to the single groove (9).

4. The atraumatic hemostatic clamp according to claim 1, wherein a width L of the jaws (3) is in a range of 0.4 cm to 0.7 cm.

5. The atraumatic hemostatic clamp according to claim 1, wherein the handles (4) of the left and right clamp bodies (1), (2) respectively have a clamping force adjustment mechanism acting in cooperation with each other, the adjustment mechanism comprises a ratchet means (6) formed on one of the left clamp body (1) and the right clamp body (2) and a pawl (7) formed on the other of the left clamp body (1) and the right clamp body (2), and the ratchet means (6) has at least five ratchet teeth.
